(19) United States
(12) Patent Application Publication
(10) Pub. No.: US 2016/0303177 A1
(43) Pub. Date: Oct. 20, 2016

(54) NUTRITIONAL SUPPLEMENT

(71) Applicant: Erroll J. Bailey, Atlanta, GA (US)

(72) Inventor: Erroll J. Bailey, Atlanta, GA (US)

(21) Appl. No.: 14/846,557

(22) Filed: Sep. 4, 2015

Related U.S. Application Data
(63) Continuation-in-part of application No. 14/687,265, filed on Apr. 15, 2015.

(51) Int. Cl. A61K 36/15; A61K 36/82; A61K 31/198; A61K 31/197; A61K 31/7004; A61K 31/685; A61K 36/736; A61K 36/79; A61K 36/41; A61K 31/122; A61K 36/21

(52) U.S. Cl. CPC A61K 36/15; A61K 31/122; A61K 36/82; A61K 31/198; A61K 31/197; A61K 36/21; A61K 31/685; A61K 36/736; A61K 36/79; A61K 36/41; A61K 31/7004; A23L 1/3051; A23L 1/3002; A23L 1/30; A23V 2002/00

(57) ABSTRACT

The present invention is a nutritional supplement made from a combination of natural ingredients. A formulation in accordance with embodiments of the present invention comprises: L-Citrulline, Pine bark extract (such as Pycnogenol®), beetroot, beta alanine, L-Carnitine, branched chain amino acids, CoQ 10, green tea extract, alpha GPC, tart cherry extract, D-Ribose, rhodiola rosea, and shisandra berry.

### CROSS REFERENCE TO RELATED APPLICATION

This application claims the benefit of and priority from U.S. nonprovisional patent application Ser. No. 14/687,265, filed Apr. 15, 2015, the entirety of which is incorporated by reference herein.

### FIELD OF INVENTION

The invention disclosed herein relates generally to nutritional supplements, and more specifically to supplements that improve endurance, quicken recovery, improve cognition, and/or generally promote a greater sense of well-being.

### BACKGROUND

Certain nutritional supplements are administered to improve a person's physical performance while engaged in sporting activities. These supplements may function to increase endurance, quicken recovery, improve cognition, and/or generally to promote a greater sense of well-being. However, supplements in general, and pre-workout supplements in particular, are primarily designed to just boost physical performance and do so by adding large amounts of stimulants. This leads to pre-workout jitters, heart arrhythmia risks (irregular beats) and post workout crashing or fatigue. In addition, most do not provide any benefit other than a potentially better workout. They usually do not provide true heart healthy ingredients or other ingredients in high enough dosages that lead to measurable physiological benefits to the heart, blood vessels, joints, the brain or reproductive system. There is an unmet need in the market for supplements that deliver benefits to the entire body.

The content of this patent application is presented solely for the purpose of being reviewed by the United States Patent and Trademark Office for patentability of the claimed nutritional supplements. In accordance with the Dietary Supplement Health and Education Act of 1994 (DSHEA), Applicant asserts that statements made within this patent application have not been evaluated by the Food and Drug Administration. Further in accordance with DSHEA, Applicant asserts that the nutritional supplements are not intended to diagnose, treat, prevent, mitigate or cure disease.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

The present invention features nutritional supplements or formulations that may have one or more of the following benefits: improve athletic performance, increase endurance, quicken muscle recovery, promote feeling of well-being, enhance cognitive abilities, thermogenesis (fat burning), cardiac rejuvenation, mild anti-inflammatory effects that may assist in joint health, assist with mild to moderate erectile dysfunction, and the like.

In one embodiment of the present invention, the formulation comprises at least the following components: L-Citrulline , pine bark extract (such as Pycnogenol®), Coenzyme Q10 (also known as ubiquinone, ubidecarenone, etc., and sometimes abbreviated as CoQ₁₀, CoQ10, CoQ-10, etc.) and L-Carnitine.

In other embodiments, the formulation comprises two or more of the following components: L-Citrulline, pine bark extract (such as Pycnogenol®), beetroot, beta alanine (sometimes abbreviated as β-Alanine; also commercially known as CarnoSyn®), L-Carnitine, branched chain amino acids (“BCAAs”), Coenzyme Q10, green tea extract, glycerylphosphorylcholine (“alpha GPC”), tart cherry extract, D-Ribose, rhodiola rosea, and shisandra berry.

These and other aspects, embodiments, features, and advantages of the invention will become better understood with regard to the following description, appended claims and accompanying drawings.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIGS. 1-8 show components of exemplary formulations in accordance with embodiments of the present invention;

FIG. 9 shows dosage information for the exemplary formulation shown in FIG. 8 in accordance with embodiments of the present invention; and

FIG. 10 shows dosage information for the exemplary formulation shown in FIG. 7 in accordance with embodiments of the present invention.

### DETAILED DESCRIPTION

The subject matter of aspects of embodiments of the present invention is described with specificity herein to meet statutory requirements. However, the description itself is not intended to limit the scope of any patent issuing from this description. Rather, the inventor has contemplated that the claimed subject matter might also be embodied in other ways, to include different elements or combinations of elements similar to the ones described in this document, in conjunction with other present or future technologies.

The present invention describes nutritional supplements or formulations that can be administered before, during, and/or after workouts to enhance performance, increase endurance, quicken recovery, improve cognition, and generally to promote a greater sense of well-being. In some embodiments of the present invention, formulations are administered about 30 minutes to about 45 minutes before a workout session. A workout session can be any physical activity such as, without limitation, weight lifting, running, rowing, basketball, football, soccer, boxing, swimming, yoga, martial arts, construction work, manual labor and the like. In alternative embodiments, formulations can also be suitably administered to assist with intense mental exercises such as, without limitation, exam preparation, lengthy meetings, depositions, and the like.

### Exemplary Formulations and Health Benefits

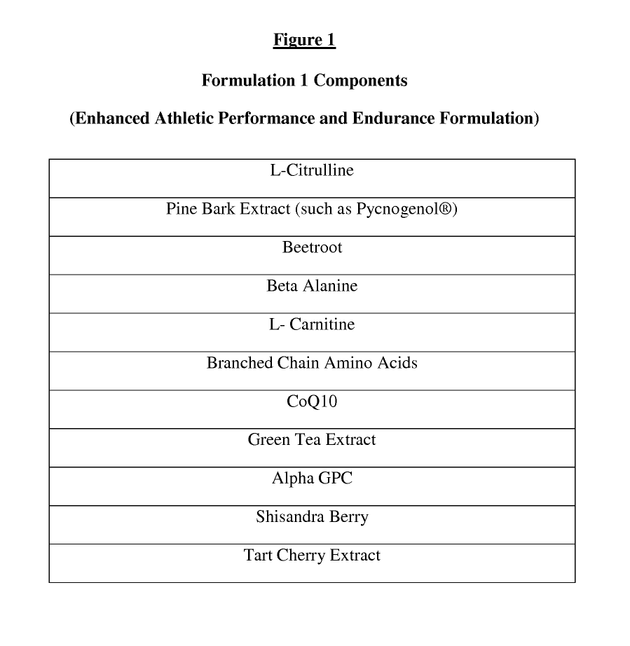

Turning now to FIGS. 1-8, these figures show eight exemplary formulations in accordance with embodiments of the present invention. Each of FIGS. 1-8 lists the constituent components of a particular exemplary formulation. Each exemplary formulation has at least one health benefit when administered. FIG. 1 shows Formulation 1 which can enhance the user's athletic performance and endurance. Formulation 1 comprises the following components: L-Citrulline, pine bark extract (such as Pycnogenol®), beetroot, beta alanine, L-Carnitine, branched chain amino acids, Coenzyme CoQ 10, green tea extract, alpha GPC, shisandra berry and tart cherry extract.

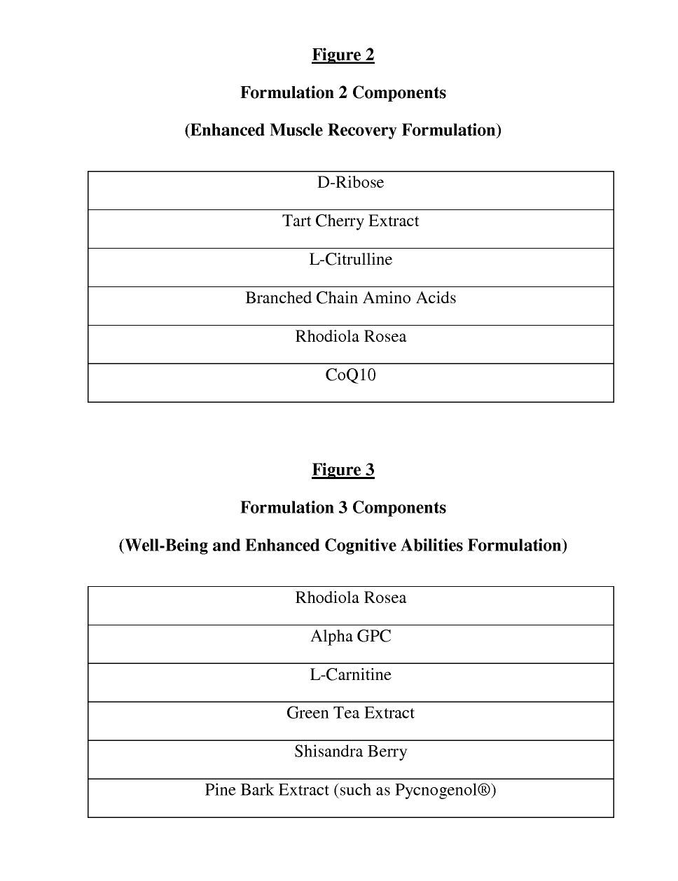

FIG. 2 shows Formulation 2 which can enhance the user's muscle recovery process. Formulation 2 comprises one or more of: D-Ribose, tart cherry extract, L-Citrulline, branched chain amino acids, rhodiola rosea, and CoQ 10.

FIG. 3 shows Formulation 3 which can increase the user's feeling of well-being and enhance his/her cognitive abilities. Formulation 3 comprises: rhodiola rosea, alpha GPC, L-Carnitine, green tea extract, shisandra berry and pine bark extract (such as Pycnogenol®). Green tea extract and L-Carnitine also promote thermogenesis (i.e., fat burning).

FIG. 4 shows Formulation 4 which can improve cardiac functions in the user. Formulation 4 comprises: CoQ 10, D-Ribose, L-Citrulline, pine bark extract (such as Pycnogenol®), tart cherry, rhodiola rosea, shisandra berry and beetroot.

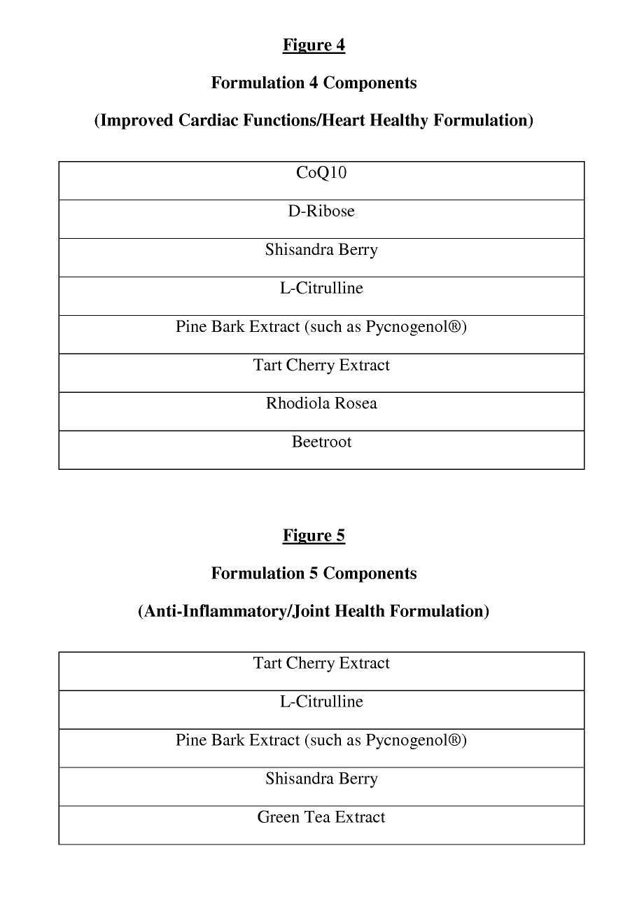

FIG. 5 shows Formulation 5 which can provide the user with mild anti-inflammatory benefits that may also assist in joint health. Formulation 5 comprises: Tart cherry extract, L-Citrulline, pine bark extract (such as Pycnogenol®), shisandra berry, and green tea extract.

FIG. 6 shows Formulation 6 which can benefit male's sexual health, or a user who suffers from mild to moderate erectile dysfunction. Formulation 6 comprises: L-Citrulline, pine bark extract (such as Pycnogenol®), shisandra berry, Co Q 10 and L-Carnitine.

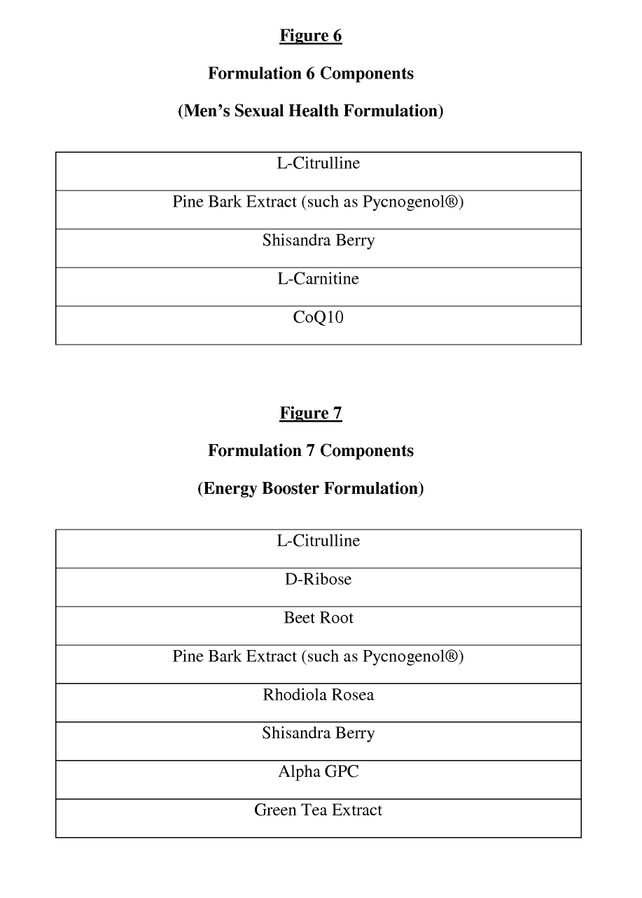

FIG. 7 shows Formulation 7 which can boost the user's energy. Formulation 7 comprises: L-Citrulline, D-Ribose, beetroot, pine bark extract (such as Pycnogenol®), Rhodiola Rosea, shisandra berry, alpha GPC, and green tea extract.

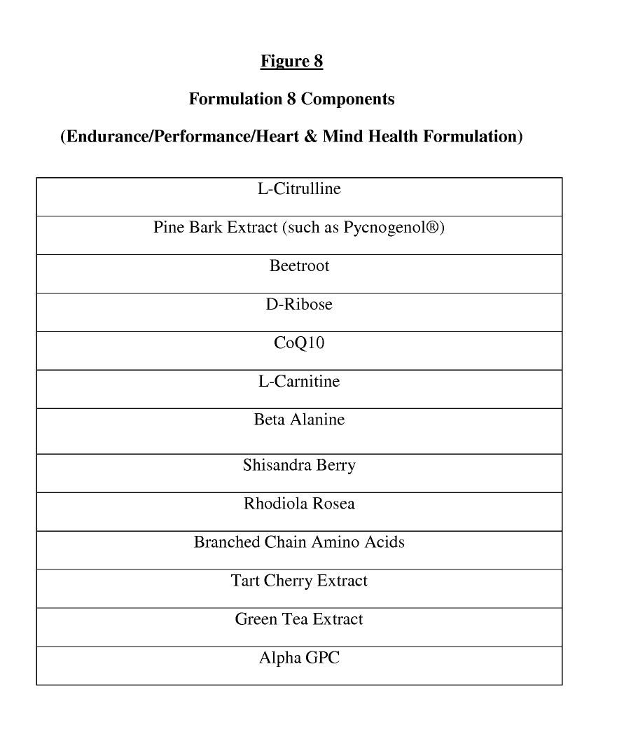

FIG. 8 shows Formulation 8 which is a super formulation that combines all the unique components found in Formulations 1-7 and thereby delivers at least all of the health benefits associated with Formulations 1-7 in one single dose. Formulation 8 comprises: L-Citrulline, pine bark extract (such as Pycnogenol®), beetroot, D-Ribose, CoQ 10, L-Carnitine, beta alanine, shisandra berry, rhodiola rosea, branched chain amino acids, tart cherry extract, green tea extract, and alpha GPC.

The user may choose to administer one or more of Formulations 1-7 to target specific health deficiencies or may administer Formulation 8 to target multiple health deficiencies at once.

### Formulation Components

FIGS. 1-8 list thirteen unique components, each of which will now be discussed in turn.

L-Citrulline is a natural amino acid found in watermelons. It is one of three dietary amino acids in the urea cycle with L-Arginine and Ornithine. Ammonia is a byproduct formed during the metabolism of protein. Since ammonia is toxic, a person's body neutralizes and eliminates it through a process called the urea cycle. The cycle follows several steps, with each one requiring specific substances to complete its part of the cycle. L-Citrulline, along with L-Arginine and Ornithine, are essential to these steps. Supplementing L-Citrulline also increases Ornithine and L-Arginine plasma content. Supplemental L-Citrulline increases arginine plasma levels over a longer period of time. Thus, L-citrulline supplementation improves the ammonia recycling process and nitric oxide metabolism. Broken down in the kidney, L- Citrulline is metabolized to L-Arginine (a potent precursor to Nitric Oxide). L-Arginine is metabolized to nitric oxide by nitric oxide synthase, and there is considerable research literature to support that nitric oxide in both men and women can cause an increase in sports performance, decrease fatigue, increase growth hormone with exercise, increase Adenosine triphosphate (ATP) production, increase blood flow, reduce muscle soreness and increase overall cardiovascular health. One study using 8 grams of L-Citrulline with 10 grams of carbohydrate showed a statistically significant delay in fatigue with an ability to do more exercise reps after the first two rounds. In other words, the participants got stronger, later in the workout. L-Citrulline (6 grams) in another study, affected skeletal muscle performance through glycogenolysis (the breakdown of glucose to ATP). It increased ATP production, reduced creatine depletion by 28%, reduced ammonia production (which otherwise would have been a toxic state), and increased muscle protein synthesis. In diseased hearts, one study showed an increase in right ventricular ejection fraction and an increase in exercise abilities at 3 grams a day. Also noted was a decrease in blood pressure. Taking L-Citrulline at 1500 mg per day for 6 weeks showed an increase in erection hardness in 50% of men with mild to moderate erectile dysfunction versus 8% in placebo.

Pine bark extract, such as, but not limited to, Pycnogenol®, is a potent antioxidant, which contains 65-75% procyanidins. Procyanidins are a subclass of flavonoids that have potential health benefits. It is believed to be helpful in treatment of circulation problems. There is supporting literature that it is a nitric oxide producer that increases blood flow and is cardio-protective. It is broken down in the colon to produce the M1 metabolite, the actual active form. Its antioxidant properties affect nitric oxide metabolism by changing nitric oxide to superoxide and thus prolonging its half-life (i.e., keeping it available at an active state longer). Studies have shown that taking pine bark extract, such as Pycnogenol®, 100 mg a day for 6 weeks resulted in a significant increase in time to fatigue on a treadmill. In addition, pine bark extract, such as Pycnogenol®, supports the heart by augmenting acetylcholine induced endothelial relaxation, thereby causing better blood flow. One study showed a 32% increase in blood flow with 200 mg for 8 weeks. It also has been shown to inhibit platelet aggregation (or clumping together of platelets in the blood which can lead to the formation of clots) by 25%. Other heart healthy benefits are an increase in high-density lipoprotein (HDL, good cholesterol) and a decrease in low-density lipoprotein (LDL, bad cholesterol). HDL reduces, reuses, and recycles LDL by transporting it to the liver where it can be reprocessed. In terms of exercise recovery, 50 mg at three times a day for 8 weeks in one study decreased joint pain and stiffness by 43% and 35%, respectively. Pine bark extract, such as Pycnogenol® inhibits pain modulator NF-KB and decreases PGF2a, thereby reducing inflammation. NF-KB is a gene that mediates major inflammatory processes inside each and every cell in the body. When this gene is turned on, high levels of inflammatory molecules are produced. PFG 2a is a type of prostaglandin that promotes many aspects of the inflammatory response. Pine bark extract, such as Pycnogenol® also has been shown to increase cognitive function, attention and mental performance in students. Pine bark extract, such as Pycnogenol® (40-80 mg) combined with arginine (1.7 g) or citrulline (1.5 g) over a 2 month period showed an 80% increase in ability to have sex in men with sexual dysfunction.

Beetroot is the taproot portion of the beet plant, also known as table beet, garden beet, red or golden beet. It is the nitrates in beetroot that provide the increase in nitric oxide giving the aforementioned benefits already stated, including a 15% increase in time to exhaustion in one study. Additionally, beetroot has a high concentration of unique phytonutrients such as betalains that contribute to increasing cardiovascular blood flow and improving endurance. Nitrates are especially abundant in beetroot. Nitrates are used up in exercise. Higher serum nitrate levels with supplementation alter the rate of nitrite decline during exercise. In particular, according to one study, nitrates seem to form a “pool” which can be used in healthy individuals to reduce oxygen costs (5.4% reduced VO₂ (or oxygen consumption) at the 0.1 mmol/kg of nitrate), increase energy efficiency by 7.1% and increase time to fatigue by 15% (at 6.2 mmol/kg of nitrate). At even higher dosing (11.2 mmol/kg of nitrate) in one study there was a 19% reduction of oxygen muscle extraction. 500 milligrams of nitrate improved the 5K time by 41 seconds in runners. Runners felt better in the first one third of the run and had increased running velocity in the final stretch. In another study involving cyclists given 500 milliters (6.2 mmol/kg) of beetroot, there was a 2.8% improvement in the first 4 kilometers and a 2.7% improvement after 16.1 kilometers, and there was also an increase in muscular power output. Overall, the 5 kilometer trial run was completed 41 seconds quicker in the group of individuals that consumed 500 milliliters of beetroot juice.

D-Ribose is a five-carbon sugar, organic compound that forms part of the backbone of ribonucleic acid (RNA). Once phosphorylated by cells it is ready for amino acid production, the pentose phosphate pathway and ATP (adenosine triphosphate) production. D-Ribose has the important function of producing ATPs which are the building blocks of energy production and muscle function. ATP is formed by the union of the nitrogenous base adenosine with D-Ribose via a phosphate group. When ATP is used in energy production it loses a phosphate group to form AMP (monophosphate) which can be replenished by a number of various enzymes. ATP at times is degraded to IMP (inosine-5-monophosphate) which does not contribute to energy production This compound is often later kicked out of the cell and eliminated. But, if IMP is kept inside the cell, it can be restored to ATP for renewed energy usage. D-Ribose is involved, with various enzymes, in the conversion of IMP back to ATP. D-Ribose is useful where ATP concentrations (relative to total nucleotides) are reduced such as in cases of cardiac disease, chronic fatigue syndrome, fibromyalgia and prolonged exercise. D-Ribose availability may increase ATP production in the cell where processes for replenishing are usually slow. D-Ribose has been found to restore glycogen as well for energy usage. Glycogen is a carbohydrate that is a fuel source for the muscles. One study involving the usage of 17.25 grams per day in cyclists increased the rates of ATP production back to pre-exercise levels. In fibromyalgia cases, where there is perturbed muscle function (i.e., reduced ATPs), 5 grams at three times a day along with other medications led to a marked decrease in symptoms. When D-Ribose was stopped, the symptoms returned in one week. In animal studies, when D-ribose was given before animals were cardiac stressed, compared to placebo, those that received D-Ribose held out 25% longer before ischemia (or decreased blood flow to the heart muscle) set in. Dosages vary from 5 grams to 15 grams per day.

Coenzyme Q10 or CoQ 10 is a substance similar to a vitamin that is found throughout the body. It is said to work as an antioxidant and helps to fight free radicals, reducing stress and toxins in the body. It preserves nitric oxide, increases blood flow and protects blood vessels by increasing endothelial function (or functioning of the inner lining of the blood vessels). CoQ 10 has a role in ATP production and thus may be energy producing via that mechanism. Low CoQ 10 is seen in fibromyalgia cases, post heart attack and in people who use statins (cholesterol reducing drugs). Chemical indicators of muscle damage (such as the presence of creatine kinase, white blood cells, and myoglobin) were reduced in athletes training 5.5 hours per day when on CoQ 10 300 mg per day. CoQ 10 imparts stability to the membranes of skeletal muscle cells and reduces the release of muscle cell by-products that lead to fatigue. The same dosage was also shown to reduce fatigue during the late portions of exertion. In sick hearts it increases left ventricular ejection fractions (ejection fraction is a measurement of the percentage of blood leaving your heart each time it contracts) and reduces cardiomegaly (enlarged heart). Its half-life is 5.8-8.10 hours and supplementation of CoQ 10, such as with 90-120 mg yields a 50-150% increase in CoQ10 concentrations. It is present in all skeletal muscle and its presence correlates with muscle oxygen capacities and aerobic exercise performance. Taking CoQ 10 orally improves physical performance in patients with muscular dystrophy and other muscular disorders. Although studies do not show profound increase in efficiency with usage of CoQ 10 supplementation in cardiovascular exercise, there is a small benefit in prolonged exercise and a small increase in power output. Its positive benefit in muscle recovery is its most consistent finding in normal skeletal muscle exercise research.

L-Carnitine is an antioxidant formed by the kidney and the liver from two amino acids, lysine and methionine. It is needed by all cells to convert fatty nutrients into energy by increasing the mitochondria's potential to burn fat (mitochondria is the cell's structure which generates energy). Additional potential benefits include free radical reduced oxidative stress (which counters aging and chronic disease), reduction of the toxin ammonia, an increase in blood flow secondary to nitrates, increased insulin sensitivity (yielding lower fasting blood sugars), decrease in heart rate, decrease in the rate of perceived exertion, reduction of fatigue (elderly individuals with low endurance) and also improvements in chronic fatigue syndrome. Supplementation of L-Carnitine in humans increases muscle carnitine levels and has been shown to reduce muscle damage and soreness, mildly reduce inflammatory markers in serum and also decrease exercise-induced oxidation. Two grams of L-Carnitine with 80 mg of carbohydrates produced a 21% increase in muscle carnitine content and increased fat burning. Combined with Propionyl L-Carnitine, arginine and niacin, it potentially can increase the strength of erections in the penis. A study in the International Journal of Cardiology in February 2006 found that cells that line blood vessels produce substances that protect against free radicals (toxins) when exposed to L-Carnitine. Other studies in aged animals showed that supplementation improved energy production by the mitochondria. It is the energy production that L-Carnitine provides in the work-out that burns the fat, unless the subject is deficient in it. Another study noted increased performance of memory tasks in animals. When combined with CoQ 10 it has a further positive effect on heart health.

Beta-Alanine is a modified version of the amino acid alanine and is synthesized in the liver and transported to muscle cells (type II muscles more than type I) where it is turned into carnosine. It is commonly found in beef, pork, chicken and fish. Higher levels are seen in wild animals. It is a building block of carnosine, a molecule that helps to buffer acid in muscles, increasing physical performance in the 60-240 second range. It is effective in improving moderate to high intensity cardiovascular performance like rowing or sprinting. It also helps with lean mass gains. Carnosine appears to be an anti-oxidant and anti-aging compound. It is stored in cells and released in response to drops in pH and offers protection from exercise-induced lactic acid production. Research studies seem to indicate that its buffering acidity activity in vivo leads to subsequent increases in performance in short term high intensity exercise via direct or indirect mechanisms. Beta-Alanine is more effective than carnosine itself at the same dosage because of absorption, and it is better absorbed with meals. It has a long washout period and may still be in the muscles at higher levels 8 weeks after supplementation. (Washout period is a period in a clinical study during which subjects receive no treatment for the indication under study and the effects of a previous treatment are eliminated (or assumed to be eliminated)). Muscle carnosine stores can be depleted in vegetarians. Weight lifters report being able to perform one to two additional reps in the gym when training in set of 8-15 repetitions. Supplementation with Beta-Alanine is not time-dependent in relation to exercise but is used as a pre-workout ingredient. It is effective in reducing fatigue, reducing fat mass, increasing lean mass, protecting nerves and sensitizing contractile muscles to calcium. The benefits of Beta-Alanine are highly associated with how much Beta-Alanine and carnosine are present in a muscle cell prior to contraction. Due to its buffering, it can reduce acidosis without affecting oxygen uptake. Despite increasing performance and lean mass, studies fail to show increase in VO₂ max, which is good. (VO₂ max is a measure of the maximum volume of oxygen that an athlete can use. It is measured in milliliters per kilogram of body weight per minute (ml/kg/min)). Performance improvements seem to be non-specific when considering demographics (e.g., footballers, wrestlers, elite rowers, etc., all benefit). There is no real synergism of Beta-Alanine with creatine although both work individually to improve performance.

Shisandra berry, like rhodiola below, is a powerful antioxidant and adaptogen, which means that it helps one adapt to his or her environment allowing one to better handle mental and physical stress. It comes from a wood climbing vine which typically produces 5 to 8 mm size berries. In China it is said to be the most protective of all herbs and plants. Due to its fatigue fighting properties, it is referred to in Chinese medicine as ‘Qi’ invigorating in the ‘Yang’ family of herbs—‘Qi’ referring to the abstract concept of vital energy and ‘Yang’ referring to the overall manifestation of bodily functions. It serves as a central nervous system stimulant without the nervous jitters of caffeine. It boosts nitric oxide levels in the body through its positive effects on the nitric oxide synthase enzyme and increases energy at a cellular level in the mitochondria. In China, it is taken abundantly by students because it can raise glutathione (an antioxidant) levels in the body, which detoxifies one's system in a way that enhances mental clarity. It also reduces anxiety, depression and mood swings. Shisandra is best known for its ability to protect the liver due to it containing special chemicals, “lignans,” which help maintain liver function, improve regeneration and reduce damage. It is often used to treat Hepatitis C. It is also helps the heart and blood vessels by causing vasodilatation, thus lowering blood pressure and increasing circulation. These effects also contribute to its potential claimed benefits on erectile dysfunction. It has been used for centuries in Russia, China, Japan and Korea to improve stamina, energy, mental clarity and circulation. Dosage can range from 500 mg to 1000 mg.

Rhodiola Rosea is sometimes called the artic root or golden root. It is an adaptogen, which means it acts in non-specific ways to increase resistance to stress without disturbing normal biological functions. Its active ingredients on a molecular basis are tyrosol and salidroside, and it is also a high source of procyanidins like Pycnogenol® and green tea. It has been used in the treatment of adrenal fatigue syndrome. Rhodiola and its properties have been studied extensively in scientific literature in France, Sweden, Germany, Norway, and Russia. In Russia it is still widely used as a remedy for fatigue, attention deficit disorder and decreased memory. In Scandinavian countries it has been used to increase the capacity for mental work, boost general strength and also improve vitality. A small study at UCLA in 2008 reported a significant improvement in 10 people with anxiety who took rhodiola for 10 weeks. Rhodiola's efficacy was affirmed in a 2011 review of 11 placebo-controlled human studies of moderate to good quality in which it was found to have beneficial effects on physical performance, mental performance, anxiety and depression with minimal side effects. In regards to fatigue, rhodiola seems to be able to significantly reduce the effects of prolonged or minor physical exhaustion that result in fatigue. This is more related to a stress and “burnout” effect, or prolonged but low intensity physical exercise. Studies show a reduction in muscle damage indicated by drops in creatine kinase, a byproduct of muscle stress. Rhodiola seems to be highly reliable in reducing fatigue symptoms and improving symptoms of stress (or enhancing well-being) in persons fatigued from non-exercise related stressors. It is also shown to improve cognitive functioning in people who experience a reduction in fatigue. In the brain it increases serotonin and reduces corticosteroids. Preliminary non-mammalian studies show evidence suggesting up to a 20% increase in lifespan secondary to mechanisms independent of caloric restriction.

Branched Chain Amino Acids (BCAAs) are three amino acids with similar structures that beneficially influence muscles. They routinely can be found in proteins or meat. They have been shown to be helpful in exercise when taken at specific times. The BCAAs are Leucine, Isoleucine and Valine. BCAA supplementation can promote muscle protein synthesis (leucine), increase glucose uptake into the cell (isoleucine), and replace the loss of BCAAs that occurs during exercise. They are important on a daily basis, and if a person takes in a very high protein diet, supplementation may not be necessary. Research studies show an increase in aerobic exercise with an increase to time to exhaustion, increased fat oxidation and a decrease in fatigue. BCAA stores in the body are under tight regulation and decrease during exercise. With high doses of Citrulline, BCAA stores seem to burn faster as if Citrulline taps into them better which may explain some of the benefits of Citrulline on exercise endurance. There is a high concentration of BCAA in skeletal muscle. A central hypothesis of fatigue is that during exercise, elevation of serotonin occurs. The plasma ration of tryptophan and L-Tyrosine (aromatic amino acids) to BCAAs (long chain neutral amino acids) is altered in favor of the aromatic ones secondary to BCAAs being destroyed. This increase in tryptophan transport to the brain (which produces serotonin via 5-HTP) leads to elevated serotonin and subsequent fatigue in rats. Supplementing BCAAs can then correct the ratio and reduce tryptophan production and lower serotonin, hindering the onset of fatigue. In terms of performance, Leucine is able to activate a protein known as “target of rapamycin” otherwise known as TOR which stimulates muscle protein synthesis. BCAAs also reduce glycogen depletion and ammonia production. For trials that are prolonged and involve outdoor activities (hiking, skiing, sailing), BCAA supplementation in high dosages (over 50 g over several hours) show some but small gains in reduction of physical and mental fatigue. It helps marathon runners as well, but slower runners do better with BCAA supplementation than faster runners. BCAAs also seem to preserve cognitive skills later in exercise and reduce neural fatigue.

Tart Cherry extract is another powerful antioxidant with profound anti-inflammatory effects (particularly gout but also for osteoarthritis) and muscle recovery effects. The productive agents in tart cherries are anthocyanins, an antioxidant found in red and purple fruits, raspberries and blueberries. Tart cherries however have the highest concentrations and have been shown to increase the activity of the body's key antioxidant enzyme, superoxide dismutase. In addition to strong anti-inflammatory properties, they also aid in muscle recovery. In one study, runners who drank 710 ml of tart cherry juice, providing at least 80 mg of various anthocyanins, daily for one week prior to and during a 24 hour relay race experienced substantially less post-race pain, compared to controls. Another group of runners who drank tart cherry juice daily from 5 days before until 2 days after a marathon had significantly reduced inflammation markers (interleukin-6 and C-reactive protein). They also recovered isometric strength faster. In another study, in young men who normally rarely exercise, drinking 12 ounces of tart cherry juice twice a day for 8 days resulted only in a 4% decrease of arm strength after repeated arm exercises-compare to a 22% decrease in strength in controls. Recovery has also been shown to be faster in leg exercises. A study at Boston University Medical Center with 663 patients found that 10 cherries a day had a 50% reduction in the incidence of gout attacks. In 2010, at the European League Against Rheumatism, led by Robert Wood Johnson researchers, it was reported that one tablespoon of tart cherry extract had a similar response. Another study at the Philadelphia VA Medical Center (published in the Arthritis and Rheumatism Journal supplement) studied at 53 patients with osteoarthritis and found a statistically significant reduction in pain stiffness and physical function when taking two 8 ounce bottles of cherry juice a day for 6 weeks. Research shows it blocks Interleukin-6 and Cox 1's and Cox 2's thereby reducing inflammation.

Green Tea Extract is another potent anti-inflammatory that also increases thermogenesis, which means it is a fat burner (but without the jitters and side effects). In addition, it has been implicated in benefiting almost every organ system in the body. It is said to be heart protective, anti-diabetic, anti-obesity, anti-carcinogenic and blood vessel protective. It contains small amounts of caffeine and theanine, a relaxing amino acid. Green tea contains catechins (compounds that affect lipid oxidation and fat burning related pathways) of which the most potent is EGCG or epigallocatechin gallate, also known as epigallocatechin-3-gallate. Positive changes in VO₂ max have been noted as well as increases in subjective well-being. Catechins tend to be poorly absorbed and serum levels after supplementation is dose related up to about 800 mg of EGCG, then they go up more dramatically. The caffeine in green tea works synergistically with EGCG to burn fat. When taken with a meal, there seems to be no changes in overall metabolic rates from green tea (although trending toward significance) but a greater proportion of energy comes from dietary fat rather than carbohydrate after 300 mgs of EGCG. The greater fat oxidation rate does not appear to occur during exercise at low dosages (270 mgs) and may be vicariously through green tea being able to inhibit carbohydrate digestion. The theory is somewhat backed up by green tea losing its effectiveness as a fat burning agent when on a high protein diet, as there are less carbohydrate (relative to adequate protein diets) to block. Of note is that catechins also are said to increase nitric oxide availability and in mice increases the time to exhaustion during endurance events. This, however, is thought to be secondary to increased mobilization of intramuscular fatty acids. Supplementation with fish oil, quercetin and COQ 9 (a metabolite of COQ 10) individually enhances the anti-oxidant potential of EGCG. In terms of potential negative interactions, a few case studies do exist in the dosage range of 10-29 mg/kg bodyweight (681-1997 mg for a 150 lb person) which tend to (8 or 9 cases) have been associated with elevated ALT and bilirubin, indicative of liver damage. Causation was placed on dietary supplement in these cases (due to symptom appearing upon reintroduction) but the authors could not rule out possible tampering of the supplements.

Alpha Glycerylphosphorylcholine (“Alpha GPC”) is a cholinergic compound (precursor to acetylcholine) that comes from lecithin and is found in a variety of foods, but usually deficient in the daily intake of humans. It is the best known cholinergic in increasing plasma and brain choline levels, as it has better transportation into the brain than does choline. It has cognitive-promoting properties and enhances power output in athletes. It appears to also support cellular membranes, and research seems to indicate that it helps counter cognitive decline. Studies show that it can enhance growth hormone production (but only acute spikes have been reported thus far). One pilot study showed that 600 mg of Alpha GPC prior to exercise showed a 14% peak increase in peak force production during resistance exercise in men. Choline also has been shown to enhance the uptake of L-Carnitine into muscle cells, possibly increasing the muscle benefits of L-Carnitine. Double-blind studies also show cognitive improvement in mild to moderate Alzheimer's dementia at 400 mg at three times per day. One study using Alpha GPC alongside both caffeine and phospatidylserine has found increased attention and reaction time in persons undergoing acute stress. Therapeutic dosages start at 300 to 600 mg.

### Formulation Dosages and Administration

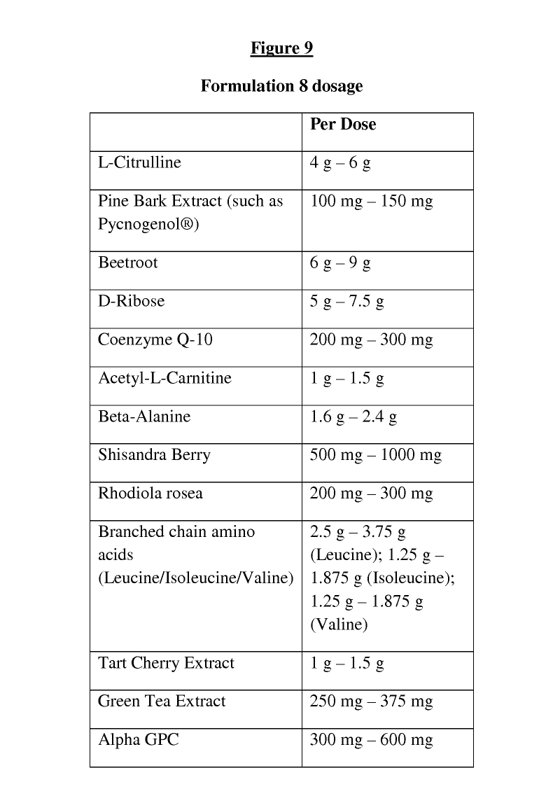

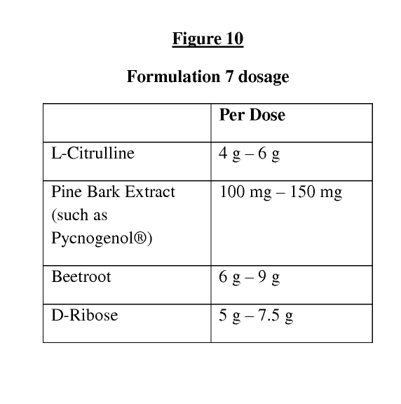

Turning now to FIG. 9, which shows the dosage amounts for exemplary Formulation 8 shown in FIG. 8. A preferred dose of Formulation 8 comprises: L-Citrulline at about 4 g to about 6 g per dose; pine bark extract (such as Pycnogenol®) at about 100 mg to about 150 mg per dose; beetroot powder at about 6 g to about 9 g per dose; D-Ribose at about 5 g to about 7.5 g per dose; Co Q 10 (Coenzyme Q-10) at about 200 to about 300 mg per dose; Acetyl-L-Carnitine at about 1 g to about 1.5 g per dose; beta Alanine at about 1.6 g to about 2.4 g per dose; shisandra berry at about 500 mg to about 1000 mg per serving; rhodiola rosea (comprising an exemplary combination of 2-3% rosavin and 0.8 -1% salidroside) at about 200 to about 300 mg per dose; BCAA comprising Leucine/Isoleucine/Valine at about 2.5 g/1.25 g/1.25 g ratio to about 3.75 g/ 1.875 g/1.875 g ratio, respectively; tart cherry extract at about 1 g to 1.5 g per dose; green tea extract at about 250 mg to about 375 mg per dose (note: a special formula with about 50% caffeine produces about 125 mg of caffeine from a dosage of about 250 mg of green tea extract); and Alpha GPC at about 300 mg to about 600 mg per dose.

For each of the exemplary formulations shown in FIGS. 1-7, the amount of each component in one dose is the same as that listed in FIG. 9. For instance and with reference to FIG. 10, the exemplary formulation shown in FIG. 7 comprises the following amounts per dose: about 4 g to about 6 g of L-Citrulline, about 100 mg to about 150 mg of pine bark extract (such as Pycnogenol®), about 6 g to about 9 g of beetroot, and about 5 g to about 7.5 g of D-Ribose.

In accordance with some embodiments of the present invention, the dosage form of the exemplary formulations shown in FIGS. 1-8 is powder. Dosage forms of alternative embodiments may be, without limitation, capsule, liquid solution, liquid suspension, chewable solid, pill, tablet, thin film, paste, gel, mist, patch and/or cream. Liquids for oral use are prepared at room temperature by dissolving prescribed quantities of the formulation ingredients in water, adding preservative and coloring and/or flavoring, filter sterilizing, and bottling. If the material is to be dispensed in a multi-dose vial, preservative is added before the pH is adjusted with NaOH to neutrality and the solution is filter sterilized and bottled. Dry forms of the formulation are prepared by mixing prescribed amounts of the dry ingredients. If the invention is to be encapsulated, an anticaking agent to facilitate production may be added prior to encapsulation.

In accordance with some embodiments of the present invention, the exemplary formulations shown in FIGS. 1-8 are administered orally. Alternative embodiments may be administered using other methods including, without limitation, topical (skin), transmucusal (e.g., nasal, vaginal, rectal, etc.), inhalation, intravenous, inhale, and/or injection. Embodiments in accordance with the present invention can be administered daily according to a regimen that spans weeks, months, or even years. Alternatively, embodiments of the present invention can be administered on a one-off basis to deliver to the user a single shot of health benefits.

In accordance with some embodiments of the present invention, the frequency with which exemplary formulations shown in FIGS. 1-8 are administered depends on the user's activity level. For instance, one dose per day may suffice when a formulation is used as a pre-workout supplement. Alternatively, half a dose per day may suffice for a less active user who uses a formulation as a daily energy supplement.

As used herein, the term “about” refers to plus or minus 10% of the referenced number. For example, a formulation comprising green tea extract at about 250 mg includes an amount of green tea extract between 225 and 275 mg.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A formulation comprising:
(i) L-Citrulline; and
(ii) Pine bark extract.

2. The formulation of claim 1 further comprising: L-Carnitine and CoQ10.

3. The formulation of claim 2 further comprising: beetroot, beta Alanine, branched chain amino acids, green tea extract, alpha GPC, and tart cherry extract.

4. The formulation of claim 3 further comprising: D-Ribose, shisandra berry, and rhodiola rosea.

5. The formulation of claim 4 wherein the formulation is in a powder form.

6. The formulation of claim 4 wherein the formulation is in a liquid form.

7. The formulation of claim 1 wherein the pine bark extract is Pycnogenol®.

8. The formulation of claim 1 further comprising: CoQ10, D-Ribose, shisandra berry, tart cherry extract, rhodiola rosea, and beetroot.

9. The formulation of claim 1 further comprising: beetroot, D-Ribose, rhodiola rosea, alpha GPC, and green tea extract.

10. The formulation of claim 1 further comprising: shisandra berry, green tea extract, and tart cherry extract.

11. A formulation comprising:
(i) Pine bark extract;
(ii) L-Carnitine;
(iii) Rhodiola rosea;
(iv) Green tea extract;
(v) Alpha GPC; and
(vi) Shisandra berry.

12. The formulation of claim 11 wherein the formulation is in a powder form.

13. The formulation of claim 11 wherein the formulation is in a liquid form.

14. The formulation of claim 11 wherein the pine bark extract is Pycnogenol®.

15. A formulation comprising:
(i) L-Citrulline;
(ii) CoQ10;
(iii) Rhodiola rosea;
(iv) Branched chain amino acids;
(v) Tart cherry extract; and
(vi) D-Ribose.

16. The formulation of claim 15 wherein the formulation is in a powder form.

17. The formulation of claim 15 wherein the formulation is in a liquid form.
